(19) United States
(12) Patent Application Publication
(10) Pub. No.: US 2009/0270620 A1
(43) Pub. Date: Oct. 29, 2009

(54) Processes for preparing crystal forms of 9-hydroxy-risperidone (paliperidone)

(76) Inventors: Santiago Ini, Haifa (IL); Naama Chasid, Peduel (IL); Ron Bar-shavit, Ariel (IL); Ronen Levinshtein, Petach-Tikva (IL); Eli Lancry, Modiin (IL)

Correspondence Address: KENYON & KENYON LLP, ONE BROADWAY, NEW YORK, NY, 10004, US

(21) Appl. No.: 12/313,964

(22) Filed: Nov. 26, 2008

Related U.S. Application Data
(60) Provisional application No. 60/990,444, filed on Nov. 27, 2007.
(60) Provisional application No. 61/041,813, filed on Apr. 2, 2008.
(60) Provisional application No. 61/052,448, filed on May 12, 2008.

(51) Int. Cl. C07D 471/04

(52) U.S. Cl. 544/282

(57) ABSTRACT

The present invention is related to processes for the preparation of paliperidone crystalline Form II. The invention also provides a paliperidone crystalline Form II containing isopropyl alcohol, and processes for preparing the same.

### CROSS-REFERENCE TO RELATED APPLICATIONS

This application claims the benefit of U.S. Provisional Application Nos. 60/990,444, filed Nov. 27, 2007; 61/041,813, filed Apr. 2, 2008; and 61/052,448, May 12, 2008, hereby incorporated by reference.

### FIELD OF INVENTION

The present invention is related to processes for the preparation of crystalline 9-hydroxy-risperidone (paliperidone).

### BACKGROUND OF THE INVENTION

RISPERDAL® (risperidone) is a psychotropic agent belonging to the chemical class of benzisoxazole derivatives. The chemical designation is 3-[2-[4-(6-fluoro-1,2-benzisoxazol-3-yl)-1-piperidinyl]ethyl]-6,7,8,9-tetrahydro-2-methyl-4H-pyrido[1,2-a]pyrimidin-4-one.

Risperidone is a selective monoaminergic antagonist which has affinity for serotonin-5-HT₂, dopamine-D₂, H₁-histamine, alpha 1- and alpha 2-adrenergic receptors. Risperidone has no affinity for cholinergic receptors. It is a potent D₂-antagonist. This active pharmaceutical ingredient is metabolized by cytochrome P-450 IID6 to produce 9-hydroxy-risperidone, also known as paliperidone, which has a similar pharmacological activity to risperidone.

Paliperidone, 3-[2-[4-(6-fluorobenzo[d]isoxazol-3-yl)-1-piperidyl]ethyl]-7-hydroxy-4-methyl-1,5-diazabicyclo[4.4.0]deca-3,5-dien-2-one, is a 5-HT antagonist belonging to the chemical class of benzisoxazole derivatives and has a racemic mixture of the following structural formula:

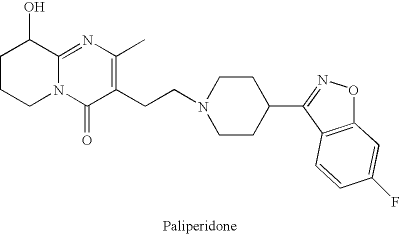

Paliperidone is a metabolite of risperidone. Marketed under the trade name, Invega®, paliperidone is a psychotropic agent approved in the United States for the treatment of schizophrenia.

Processes for the synthesis of paliperidone are described in U.S. Pat. No. 5,158,952, U.S. Pat. No. 5,254,556 and WO 96/23784.

WO 08/021342 describes several crystalline forms of paliperidone including form II, IV and V. They are characterized by X-ray diffraction (XRD) and NMR. The disclosures of WO 08/021342 are herein incorporated by reference.

Crystalline Form II of paliperidone is characterized by a powder XRD pattern having peaks at about 10.3, 14.6, 22.0, 24.6 and 25.0±0.2 degrees 2-theta according to WO 08/021342.

Crystalline paliperidone Form IV is characterized by a powder XRD pattern having peaks at about 10.2, 12.2 and 15.5±0.2 degrees 2-theta according to WO 08/021342.

Crystalline paliperidone Form V is characterized by a powder XRD pattern having four or more peaks from the list of about 9.8, 10.9, 15.8, 21.2 and 21.6±0.2 degrees 2-theta according to WO 08/021342.

The present invention relates to processes for preparing the crystalline forms of paliperidone.

The present invention also relates to crystalline forms of paliperidone containing a solvent. When a substance crystallizes out of solution, it may trap molecules of solvent at regular intervals in the crystal lattice. Solvation also affects utilitarian physical properties of the solid state like flowability and dissolution rate.

There is a need in the art for additional processes for preparing polymorphic forms of 9-hydroxy-risperidone (also referred to as paliperidone).

### SUMMARY OF THE INVENTION

In one embodiment, the present invention provides a process for preparing paliperidone crystalline Form II comprising slurrying a mixture of crystalline Forms II and V of paliperidone in at least one solvent selected from the group consisting of: C₂-C₅ alcohols, C₄-C₅ ethers, C₃-C₆ ketones, C₆-C₁₀ aromatic hydrocarbons, C₃-C₆ esters, C₁-C₆ chlorinated aliphatic hydrocarbons and C₆-C₁₀ chlorinated aromatic hydrocarbons.

In another embodiment, the present invention provides a process for preparing paliperidone crystalline Form II, comprising drying a mixture of crystalline Forms II and IV of paliperidone, under atmospheric pressure.

In another embodiment, the present invention provides a process for preparing paliperidone crystalline Form II comprising slurrying paliperidone crystalline Form IV in at least one solvent selected from the group consisting of: C₂-C₅ alcohols, C₄-C₅ ethers, C₃-C₆ ketones, C₆-C₁₀ aromatic hydrocarbons, C₃-C₆ esters, C₁-C₆ chlorinated aliphatic hydrocarbons and C₆-C₁₀ chlorinated aromatic hydrocarbons.

In one embodiment, the present invention provides a paliperidone crystalline Form II containing isopropyl alcohol (IPA), wherein the powder X-ray diffractogram (XRD) of the paliperidone crystalline Form II containing IPA is the same as the powder XRD of the paliperidone crystalline Form II.

In another embodiment, the present invention provides a process for preparing a paliperidone crystalline Form II containing IPA comprising slurrying a mixture of paliperidone crystalline Forms II and V in IPA.

In another embodiment, the present invention provides a process for preparing a paliperidone crystalline Form II containing IPA comprising slurrying paliperidone crystalline Form IV in IPA.

### DETAILED DESCRIPTION OF THE INVENTION

As used herein, unless otherwise defined, “room temperature” refers to a temperature of about 15° C. to about 35° C. Preferably, the room temperature is a temperature of about 20° C. to about 25° C.

As used herein, unless otherwise defined, the term “atmospheric pressure” refers to the standard pressure of 1±0.1 atm or 760±76 mm Hg (torr).

As used herein, unless otherwise defined, the term “overnight” refers to a period of time of about 8 to about 20 hours. Preferably, an “overnight” period is a period of about 17 hours to about 20 hours, e.g., about 17 hours or about 20 hours.

As used herein, unless otherwise defined, the term “slurrying” refers to stirring at least one solid in a liquid. During the slurrying operation, there may be some dissolution of the solid in the liquid, but there is no complete dissolution of the solid in the liquid.

As used herein, the term “GC measurement of residual solvent” refers to an automatic headspace gas-chromatographic system.

As used herein, the term “Form II containing IPA” is meant to include Form II which incorporates IPA in a level of more than about 0.5%.

In one embodiment, the present invention provides a process for preparing paliperidone crystalline Form II comprising drying a mixture of crystalline Forms II and IV of paliperidone weighing about 800 g or less under atmospheric pressure.

Preferably, the drying is performed at a temperature of about room temperature to about 140° C. More preferably, the drying is performed at a temperature of about 50° C. Even more preferably, the drying is performed overnight.

In another embodiment, the present invention provides a process for preparing paliperidone crystalline Form II comprising slurrying a mixture of crystalline Forms II and V of paliperidone in at least one solvent selected from the group consisting of: C₂-C₅ alcohols, C₄-C₅ ethers, C₃-C₆ ketones, C₆-C₁₀ aromatic hydrocarbons, C₃-C₆ esters, C₁-C₆ chlorinated.aliphatic hydrocarbons and C₆-C₁₀ chlorinated aromatic hydrocarbons.

Typically, the mixture of crystalline Forms II and V of paliperidone and the at least one solvent is stirred, the stirred mixture is then cooled to obtain a wet cake of paliperidone. The wet cake of paliperidone is subsequently filtered and dried to provide crystalline Form II of paliperidone.

Preferably, the mixture of paliperidone crystalline Forms II and V is obtained by drying a mixture of paliperidone crystalline Forms IV and II, wherein the mixture weighs about 1 kg or more. The mixture of paliperidone crystalline Forms IV and II can weigh about 10 kg to about 40 kg, or about 20 kg to about 30 kg. For example, when a mixture of paliperidone Forms IV and II weighing about 1 Kg or more is dried at about 55° C.-65° C., a mixture of paliperidone Forms V and II is obtained.

For the most part, when drying a mixture of Forms II and IV weighing about 800 g or less results in paliperidone crystalline Form II as the product. When drying a mixture of paliperidone Forms II and IV weighing about 1 kg or more results in a mixture of paliperidone Forms II and V as the product.

Preferably, the C₂-C₅ alcohol is ethanol, isopropyl alcohol (IPA) or butanol.

Preferably, the C₄-C₅ ether is diethyl ether, diisopropyl ether, methyl t-butyl ether, 1,2-dimethoxyethane, tetrahydrofuran (“THF”), methyltetrahydrofuran (“MeTHF”) or 1,4-dioxane.

Preferably, the C₃-C₆ ketone is acetone, methyl ethyl ketone (MEK) or methyl isobutyl ketone (MIBK).

Preferably, the C₆-C₁₀ aromatic hydrocarbon is benzene, toluene, o-xylene, m-xylene, p-xylene, or ethylbenzene. More preferably, the C₆-C₁₀ aromatic hydrocarbon is selected from the group consisting of toluene, o-xylene, m-xylene and p-xylene.

Preferably, the C₃-C₆ ester is ethyl acetate, propyl acetate or butyl acetate.

Preferably, the C₁-C₆ chlorinated aliphatic hydrocarbon is chloroform, dichloromethane, carbon tetrachloride or 1,2-dichloroethane.

Preferably, the C₆-C₁₀ chlorinated aromatic hydrocarbon is selected from the group consisting of chlorobenzene and dichlorobenzene.

More preferably, the at least one solvent is isopropyl alcohol (“IPA”).

In one specific embodiment, a mixture of paliperidone crystalline Forms II and V and IPA is slurried at reflux, preferably for 50 minutes, and further cooled to obtain a wet cake of paliperidone Form II. Preferably, the cooling is to a temperature of about 22° C. The wet cake is then filtered and dried in a vacuum oven under a reduced pressure of less than 100 mm Hg and at a temperature of about 50° C. to obtain paliperidone Form II.

In another specific embodiment, a mixture of paliperidone Forms IV and II is dried at about 55° C.-60° C. to obtain a mixture of paliperidone Forms V and II. The mixture of paliperidone Forms V and II is slurried in IPA at about 70° C.-80° C. for more than 1 hour and then cooled to a temperature of about 20° C.-25° C. After filtration, the resulting cake is dried at about 55° C.-65° C. to obtain paliperidone crystalline Form II.

In another embodiment, the present invention provides a process for preparing paliperidone crystalline Form II comprising slurrying paliperidone crystalline Form IV in a solvent selected from the group consisting of: C₂-C₅ alcohols, C₄-C₅ ethers, C₃-C₆ ketones, C₆-C₁₀ aromatic hydrocarbons, C₃-C₆ esters, C₁-C₆ chlorinated aliphatic hydrocarbons and C₆-C₁₀ chlorinated aromatic hydrocarbons.

Typically, the mixture of crystalline Form IV of paliperidone and the solvent is stirred, and the mixture is then cooled to obtain a wet cake of paliperidone. The wet cake of paliperidone is subsequently filtered and dried to provide crystalline Form II of paliperidone.

Preferably, the C₂-C₅ alcohol is ethanol, isopropyl alcohol (IPA) or butanol. Preferably, the C₄-C₅ ether is diethyl ether, tetrahydrofuran (“THF”) or methyltetrahydrofuran (“MeTHF”). Preferably, the C₃-C₆ ketone is acetone, methyl ether ketone (MEK) or methyl isobutyl ketone (MIBK). Preferably, the C₆-C₁₀ aromatic hydrocarbon is toluene, xylene or ethylbenzene. Preferably, the C₃-C₆ ester is ethyl acetate, propyl acetate or butyl acetate. Preferably, the C₁-C₆ chlorinated aliphatic hydrocarbon is chloroform, dichloromethane, or 1,2-dichloroethane. Preferably, the C₆-C₁₀ chlorinated aromatic hydrocarbon is chlorobenzene, or dichlorobenzene. More preferably, the solvent is isopropyl alcohol.

Preferably, the slurrying is at a reflux temperature.

Preferably, the cooling is to a temperature of about 15° C. to about 40° C. More preferably, the cooling is to a temperature of about 20° C. to about 35° C. Most preferably, the cooling is to a temperature of about 20° C. to about 25° C.

Preferably, the drying is performed at a temperature of about room temperature to about 140° C. More preferably, the drying is performed at a temperature of about 50° C. in a vacuum oven under a reduced pressure of less than 100 mmHg.

In a specific embodiment, a mixture of paliperidone Form IV and IPA is stirred at reflux, preferably for one hour, and further cooled to obtain a wet cake of paliperidone Form II. Preferably the cooling is to a temperature of about 25° C. The wet cake is then filtered and dried in a vacuum oven under a reduced pressure of less than 100 mm Hg and at a temperature of about 50° C. to obtain paliperidone Form II.

In another specific embodiment, a mixture of paliperidone Form IV and acetone is stirred at about 25° C. to about 60° C., preferably, at about 55° C. to about 60° C. and further cooled to obtain a wet cake of paliperidone Form II. Preferably, the cooling is to a temperature of about 15-40° C. More preferably, the cooling is to a temperature of about 20-35° C. Most preferably, the cooling is to a temperature of about 20-25° C., The wet cake is then filtered and dried in a vacuum oven under a reduced pressure of less than 100 mm Hg and at a temperature of about 50° C. to obtain paliperidone Form II.

In the above processes for obtaining paliperidone form II, the reactants do not achieve complete dissolution when heating to reflux.

In the above processes for obtaining paliperidone form II, depending on the time for drying the precipitate, paliperidone form II containing IPA may be obtained when using IPA as the solvent. One skilled in the art could easily monitor the reaction to determine whether a sufficient period of time has lapsed.

In one embodiment, the present invention provides a paliperidone crystalline Form II containing IPA.

Preferably, the paliperidone crystalline Form II contains about 0.5% to about 5% by weight of IPA, more preferably, it contains about 2% by weight of IPA, as measured by thermogravimetric analysis (TGA).

GC measurement of residue solvents of paliperidone crystalline Form II gave 15291 ppm of IPA.

In one specific embodiment, a mixture of paliperidone Form IV and IPA is stirred at 50° C. preferably for one hour and further cooled to obtain a wet cake of paliperidone Form II. Preferably the cooling is to room temperature. The wet cake is then filtered and dried in a vacuum oven under a reduced pressure of less than 100 mm Hg and at a temperature of about 50° C. to obtain a paliperidone Form II containing IPA.

In another embodiment, the present invention provides a process for preparing paliperidone crystalline Form II containing IPA comprising slurrying a mixture of paliperidone crystalline Forms II and V with IPA.

The starting paliperidone which is used in the above described processes may be prepared directly from the compound:

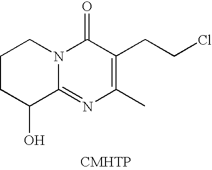

with the chemical name of 3-(2-chloroethyl)-6,7,8,9-tetrahydro-9-hydroxy-2-methyl-4H-pyrido[1,2-a]-pyrimidin-4-one (CMHTP) and from the compound:

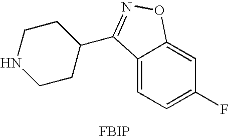

with the chemical name of 6-fluoro-3-piperidino-1,2-benzisoxazole (FBIP). The paliperidone may be prepared by combining CMHTP and FBIP in the presence of an inorganic base.

The starting paliperidone which can be used in the above described processes may be prepared by combining CMHTP, or a salt thereof, and FBIP, or a salt thereof, in the presence of an inorganic base to provide a reaction mixture, preferably in the ratio of about 1 to about 3 moles of the inorganic base per mole of CMHTP. Preferably, a solvent is also added to the reaction mixture. Preferably, the reaction is performed under nitrogen. Optionally, a phase transfer catalyst (PTC) may also be present.

The inorganic base can be potassium carbonate or, preferably, sodium carbonate.

The solvent is preferably selected from the group consisting of water, C₁-C₈ alkyl alcohols, acetonitrile, C₃-C₆ amides, C₃-C₆ ketones, C₆-C₁₂ aromatic hydrocarbons, C₂-C₆ alkyl acetates and C₄-C₈ ethers. Preferred C₁-C₈ alkyl alcohols are methanol, ethanol, n-propanol, isopropanol (IPA), n-butanol, isobutanol and 2-butanol. Preferred C₃-C₆ amides are dimethylacetamide and dimethylformamide (DMF). Preferred C₃-C₆ ketones are acetone, methyl ethyl ketone (MEK) and methyl iso-butyl ketone (MIBK). Preferred C₆-C₁₂ aromatic hydrocarbons are benzene, toluene and xylene. Preferred C₂-C₆ alkyl acetates are ethyl acetate and isobutyl acetate. Preferred C₄-C₈ ethers are tetrahydrofurane (THF), diethoxymethane (DEM), isobutyl methyl ether, dibutyl ether and polyethylene glycol (PGME). More preferably, the solvent is water, acetonitrile, IPA or DMF. Even more preferably, the solvent is selected from IPA and acetonitrile, and most preferably, the solvent is acetonitrile.

Typically, the phase transfer catalyst is selected from the group consisting of tetraalkylammonium halides, tetraarylammonium halides, and tetra(alkyl)(aryl) ammonium halides, wherein the alkyl and aryl are the same or different. Preferably the alkyl is C₁-₆ alkyl. Preferably, the aryl is C₆-₁₀ aryl. Preferably, the halide is chloride, bromide or iodide. The phase transfer catalyst is preferably selected from the group consisting of tetrabutylmethylammonium bromide and tetrabutylmethylammonium iodide.

Typically, the obtained reaction mixture is heated, preferably to a temperature of about 60° C. to about 75° C. and maintained for at least about 8 hours, for the reaction to take place.

Subsequently, the reaction mixture is cooled. Preferably, the reaction mixture is cooled to about 2° C. to about 15° C. and then to a temperature of below 0° C. Preferably, the reaction mixture is further cooled to a temperature of about −10° C.

After the cooling step, solid paliperidone is formed, which is then recovered by any known methods in the art. Preferably, the obtained paliperidone is first washed with an organic solvent, such as acetonitrile, acetone, dichloromethane or IPA, followed by drying. Preferably, the drying is performed at about 55° C. to about 65° C. More preferably, the drying is performed at about 60° C. for about 1 hour.

In order to prepare paliperidone crystalline Form IV, the paliperidone prepared from the reaction of the CMHTP and FBIP can be recrystallized from n-propanol, dioxane, an acetone/water mixture having a volume ratio ranging from about 1:1 to about 3:1, or about 3:1 to about 5:1, or methanol/water mixture having a volume ratio ranging from about 3:1 to about 5:1 as described in WO 2008/021342 to prepare the paliperidone crystalline Form IV to be used as the starting material in some of the processes of the present invention. When the paliperidone crystalline Form IV is recrystallized from n-propanol or dioxane, water can be added to induce precipitation of the paliperidone crystalline Form IV.

In another specific embodiment, the present invention provides a process for preparing paliperidone crystalline Form II comprising reacting CMHTP, FBIP, a solvent and a base to provide a reaction mixture; crystallizing the reaction mixture from acetone and water to obtain a mixture of paliperidone Forms II and IV; drying the paliperidone Forms II and IV mixture to obtain a mixture of paliperidone Forms II and V and slurrying the paliperidone Forms II and V mixture in IPA at about 70° C.-80° C. to form paliperidone crystalline Form II. Preferably, the solvent is acetonitrile and the base is sodium carbonate.

The paliperidone prepared from the reaction of the CMHTP and FBIP can also be recrystallized from an acetone/water mixture having a volume ratio of about 1:5 as described in WO 2008/021342, followed by drying to obtain a mixture of paliperidone crystalline Forms II and V to be used as the starting materials in some of the processes of the present invention.

The mixture of paliperidone crystalline Forms II and V to be used as the starting materials in some of the processes of the present invention can also be prepared by by drying a mixture of paliperidone crystalline Forms IV and II. For example, when a mixture of paliperidone Forms IV and II is dried at about 55° C.-65° C., preferably 55° C.-60° C., a mixture of paliperidone Forms V and II is obtained.

The mixture of paliperidone crystalline Forms II and IV to be used as the starting materials in some of the processes of the present invention can be prepared by reacting CMHTP, FBIP HCl in acetonitrile with sodium carbonate and dissolving the obtained product in acetone and water at reflux, and then cooled to about 1° C.-11° C. and stirred for 12 hours, filtered and washed with acetone.

While it is apparent that the invention disclosed herein is well calculated to fulfill the objects stated above, it will be appreciated that numerous modifications and embodiments may be devised by those skilled in the art. Therefore, it is intended that the appended claims cover all such modifications and embodiments as falling within the true spirit and scope of the present invention.

### EXAMPLES

### Example 1

### Preparation of Paliperidone Crystalline Form II From a Mixture of Paliperidone Crystalline Forms II and IV

A mixture of Form II and Form IV of paliperidone was dried in an oven at 50° C., overnight, under atmospheric pressure, to give paliperidone crystalline Form II.

### Example 2

### Preparation of Paliperidone Crystalline Form II From a Mixture of Paliperidone Crystalline Forms II and V

Into a 6 liter glass reactor equipped with a mechanical stirrer was charged with 72.9 g of paliperidone (crystalline Forms V and II) and 1458 ml of IPA. The slurry was stirred for 50 minutes at reflux, cooled to 22° C., filtered and washed with 100 ml IPA. The wet cake was dried in a vacuum oven under a reduced pressure (less than 100 mmHg) at 50° C. until a dried product was obtained. The resulting solid was analyzed to be paliperidone crystalline Form II according to PXRD.

### Example 3

### Preparation of Paliperidone Crystalline Form II From Paliperidone Crystalline Form IV

Into a 250 ml glass reactor equipped with a mechanical stirrer was charged with 12 g of paliperidone (crystalline form IV) and 36 ml of IPA. The slurry was stirred for 1 hour at reflux, cooled to 25° C. and filtered. The wet cake was dried in a vacuum oven under a reduced pressure (less than 100 mmHg) at 50° C. until a dried product was obtained. The resulting solid was analyzed to be paliperidone crystalline Form II according to PXRD.

### Example 4

### Preparation of Paliperidone Crystalline Form II From Paliperidone Crystalline Form IV

Into a 1 liter glass reactor with a mechanical stirrer was charged 10 g (on a dry basis) of wet paliperidone form IV and 200 ml of acetone. The slurry was stirred at reflux, 55-60° C., cooled to 20-25° C., and filtered. The wet cake was dried in a vacuum oven under a reduced pressure (under 100 mmHg) at 50° C. until a dried product was obtained. The resulting solid was analyzed to be paliperidone crystalline Form II according to PXRD.

### Example 5

### Preparation of Paliperidone Crystalline Form II Containing IPA

10.3 g (on a dry basis) of wet paliperidone form IV was charged into a 0.25 liter glass reactor equipped with a mechanical stirrer, and controlled heating/cooling system. 200 ml of IPA was charged into the reactor, the agitator was turned on, and a suspension was obtained. The jacket temperature was adjusted to 50° C. The suspension was heated and stirred for at least 1 hour, cooled to room temperature and filtered. The cake product was dried in a vacuum oven under a reduced pressure (under 100 mmHg) at 50° C. until a dried product was obtained. The resulting solid was analyzed to be paliperidone crystalline Form II according to PXRD. The resulting solid was further analyzed by GC for residual IPA and found to have 15291 ppm IPA. The resulting solid was further analyzed by TGA and showed 1.9% weight loss in the range of 26 to 185° C.

### Example 6

### Preparation of Paliperidone Crystalline Form II—Scale Up Process

192 liter of acetonitrile, 12 Kg of F-BIP HCl, 11.4 Kg of CMHTP and 10.0 Kg of sodium carbonate were charged into 400 Liter glass reactor. The suspension was mixed and bubbled with nitrogen for 3 hrs, heated to 60-75° C. and stirred at 60-75° C. until reaction completion, the reaction mixture was further cooled to 2-15° C., stirred, filtered and washed with acetonitrile. The obtained cake was suspended with 120 liter of water, filtered and washed with water until reaching neutral pH. The obtained cake (after water slurry) was dissolved in 648 liter of acetone and 216 liter of water at reflux, cooled to 1-11° C., stirred for 12 hours, filtered and washed with acetone. The obtained product (a mixture of forms IV+II) was dried at 55-65° C. The resulting dry cake (a mixture of forms V and II) was suspended in 90 liter of IPA at 70-80° C. for more than 1 hr cooled to 20-25° C., filtered and washed with IPA. The resulting wet cake was dried at 55-65° C. The final product weight was between 13.4-15.7 Kg. The resulting solid was analyzed to be paliperidone crystalline Form II according to PXRD.

We claim:

1. A process for preparing paliperidone crystalline Form II comprising slurrying a mixture of paliperidone crystalline Forms II and V in at least one solvent to obtain the paliperidone crystalline Form II, wherein the at least one solvent is selected from the group consisting of C₂-C₅ alcohols, C₄-C₅ ethers, C₃-C₆ ketones, C₆-C₁₀ aromatic hydrocarbons, C₃-C₆ esters, C₁-C₆ chlorinated aliphatic hydrocarbons and C₆-C₁₀ chlorinated aromatic hydrocarbons.

2. The process of claim 1, wherein the at least one solvent is selected from the group consisting of ethanol, isopropyl alcohol, butanol, diethyl ether, tetrahydrofuran, methyltetrahydrofuran, diisopropyl ether, methyl t-butyl ether, 1,2-dimethoxyethane, 1,4-dioxane, acetone, methyl ethyl ketone, methyl isobutyl ketone, benzene, toluene, o-xylene, m-xylene, p-xylene, ethylbenzene, ethyl acetate, propyl acetate, butyl acetate, chloroform, dichloromethane, carbon tetrachloride, 1,2-dichloroethane, chlorobenzene and dichlorobenzenes.

3. The process of claim 2, wherein the at least one solvent is isopropyl alcohol.

4. The process of claim 1, wherein the slurrying step is performed by stirring the mixture of paliperidone crystalline Forms II and V and the at least one solvent at a reflux temperature.

5. The process of claim 1, further comprising cooling the slurried mixture of paliperidone crystalline Forms II and V and at least one solvent to provide a wet cake of paliperidone; and drying the wet cake of paliperidone to provide the crystalline form II of paliperidone.

6. The process of claim 5, wherein the cooling is to a temperature of about 15° C. to about 40° C.

7. The process of claim 6, wherein the cooling is to a temperature of about 20° C. to about 25° C.

8. The process of claim 5, wherein the drying is performed at a temperature of about room temperature to about 140° C. under a reduced pressure of less than 100 mm Hg.

9. The process of claim 8, wherein the drying is performed at a temperature of about 50° C. under a reduced pressure of less than 100 mm Hg.

10. A process for preparing paliperidone crystalline Form II comprising reacting 3-(2-chloroethyl)-6,7,8,9-tetrahydro-9-hydroxy-2-methyl-4H-pyrido[1,2-a]-pyrimidin-4-one, 6-fluoro-3-piperidino-1,2-benzisoxazole, a solvent and a base to provide a reaction mixture; crystallizing the reaction mixture from acetone and water to obtain a mixture of paliperidone Forms II and IV; drying the paliperidone Forms II and IV mixture to obtain a mixture of paliperidone Forms II and V and slurrying the paliperidone Forms II and V mixture in IPA at about 70° C.-80° C. to form paliperidone crystalline Form II.

11. The process of claim 10, wherein the the solvent is acetonitrile and the base is sodium carbonate.

12. A process for preparing a mixture of paliperidone crystalline Forms II and V comprising drying a mixture of paliperidone crystalline Forms IV and II weighing about 1 kg or more.

13. The process of claim 12, wherein the drying is at a temperature of about 55° C.-60° C.

14. A process for preparing paliperidone crystalline Form II, comprising drying a mixture of crystalline Form II and Form IV of paliperidone weighing about 800 g or less, under atmospheric pressure, to form the paliperidone crystalline Form II.

15. The process of claim 14, wherein the drying is performed at a temperature of about room temperature to about 140° C.

16. The process of claim 15, wherein the drying is performed at a temperature of about 50° C.

17. A process for preparing paliperidone crystalline Form II comprising slurrying paliperidone crystalline Form IV in at least one solvent to obtain the paliperidone crystalline Form II, wherein the at least one solvent is selected from the group consisting of C₂-C₅ alcohols, C₄-C₅ ethers, C₃-C₆ ketones, C₆-C₁₀ aromatic hydrocarbons, C₃-C₆ esters, C₁-C₆ chlorinated aliphatic hydrocarbons and C₆-C₁₀ chlorinated aromatic hydrocarbons.

18. The process of claim 17, wherein the at least one solvent is selected from the group consisting of ethanol, isopropyl alcohol, butanol, diethyl ether, tetrahydrofuran, methyltetrahydrofuran, diisopropyl ether, methyl t-butyl ether, 1,2-dimethoxyethane, 1,4-dioxane, acetone, methyl ethyl ketone, methyl isobutyl ketone, benzene, toluene, o-xylene, m-xylene, p-xylene, ethylbenzene, ethyl acetate, propyl acetate, butyl acetate, chloroform, dichloromethane, carbontetrachloride, 1,2-dichloroethane, chlorobenzene and dichlorobenzenes.

19. The process of claim 18, wherein the at least one solvent is isopropyl alcohol or acetone.

20. The process of claim 17, wherein the slurrying step is performed by stirring the paliperidone crystalline Form IV and the at least one solvent at a reflux temperature.

21. The process of claim 17, further comprising the following steps: cooling the slurried mixture of the paliperidone crystalline Form IV and the at least one solvent to provide a wet cake of paliperidone; and drying the wet cake of paliperidone to provide the crystalline Form II of paliperidone.

22. The process of claim 21, wherein the cooling is to a temperature of about 15° C. to about 40° C.

23. The process of claim 22, wherein the cooling is to a temperature of about 20° C. to about 25° C.

24. The process of claim 21, wherein the drying is performed at a temperature of about room temperature to about 140° C. under a reduced pressure of less than 100 mm Hg.

25. The process of claim 24, wherein the drying is performed at a temperature of about 50° C. under a reduced pressure of less than 100 mm Hg.

26. The process of claim 1 or 17, wherein the process provides a paliperidone crystalline Form II containing isopropyl alcohol.

27. A paliperidone crystalline Form II containing isopropyl alcohol.

28. The paliperidone crystalline Form II of claim 27, wherein the paliperidone crystalline Form II contains about 0.5% to about 5%, by weight, of isopropyl alcohol.

29. The paliperidone crystalline Form II of claim 28, wherein the paliperidone crystalline Form II contains about 2%, by weight, of isopropyl alcohol.
